(19) United States
(12) Patent Application Publication
(10) Pub. No.: US 2019/0183610 A1
(43) Pub. Date: Jun. 20, 2019

(54) SELF-ILLUMINATING CHEEK RETRACTOR

(71) Applicants: SREEVATSAN RAGHAVAN, NOIDA (IN); MANVENDRA SINGH GAHLOT, SIKAR (IN)

(72) Inventors: SREEVATSAN RAGHAVAN, NOIDA (IN); MANVENDRA SINGH GAHLOT, SIKAR (IN)

(21) Appl. No.: 16/284,034

(22) Filed: Feb. 25, 2019

Related U.S. Application Data
(63) Continuation-in-part of application No. 15/544,659, filed on Jul. 19, 2017, now abandoned.

(51) Int. Cl. A61C 5/90; A61B 1/24; A61B 1/06; A61C 17/06; A61B 17/02

(52) U.S. Cl. CPC A61C 5/90; A61B 1/24; A61B 2017/00907; A61C 17/043; A61B 17/0218; A61B 1/0646

(57) ABSTRACT

A chemi-luminescent cheek retractor, wherein, the chemi-luminiscence is not localized to a specific location but the whole cheek retractor has multiple compartments to provide adequate and uniform illumination. Further, the cheek retractor is inflated with chemi-luminescent reagent and triggered by triggering agent thus allowing a chemi-luminescent reaction. The chamber is made of transparent heat resistant flexible material to withstand the heat generated during the chemi-luminescent reaction. An angular oris is lined with a reflective coating on the mouth facing surface to aid in better and uniform illumination; one detachable extendable flange is provided to abet attachments for providing multi-functionality, the purpose of detachable components is to facilitate easy cleaning and sterilization.

### CROSS REFERENCE TO RELATED APPLICATIONS

This application is a continuation-in-part of U.S. patent application Ser. No. 15/544,659 filed on Jul. 19, 2017, each hereby incorporated by reference herein. The U.S. Ser. No. 15/544,659 the United States National Stage of International Patent Corporation Treaty application no. PCT/IB2016/050244, filed Jan. 19, 2016 claiming priority of Indian Application No. 172/DEL/2015 dated Jan. 20, 2015.

### FIELD OF THE INVENTION

The present invention provides a self-illuminating cheek retractor. More particularly, the invention relates to an improved apparatus wherein the chemical luminescence is not compartmentalized and localized, rather the cheek retractor (angular oris) including the handle(s) provides light from various angles and is directed by a specialized coating, to eliminate shadowing while performing a procedure.

### BACKGROUND OF THE INVENTION

Dental operatory light (DOL) is one of the most important equipments that augment a dentist's ability to diagnose or treat patient's oral cavity. The pre requisites for good DOL are safety, energy efficiency, durability, heat management and hassle free mounting, positioning and cleaning. The conventional DOLs are designed keeping in mind that the dentist is close to the patient and shall carry various instruments therefore these lights are specifically designed to eliminate any possible shadowing that may occur in the mouth cavity during treatment. This has made it an almost universal practice for the dentist to employ movable light sources comparatively close to the patient. Therefore, DOL are prominently mounted for suitable reach and configuration.

Most of the times, it is also a desirable practice to swing the chair to some extent so that high intensity of illumination is incident onto the surfaces on which the dentist is working, so that operations are done under adequate lighting; all of which must be received through the comparatively small opening of the mouth.

The main drawback of these conventional DOL are that they are large and bulky are cluttered up for space, therefore constant adjustment is required; moreover constant adjustment of the dental chair is also required each time a patient moves. Such adjustments take the attention of the dentist away from the more important work in hand, are unsanitary, and annoying to the patient. All this is done to get the best incidence of the light. In addition to the above drawbacks, since the lights are bright and directed towards the face of a patient for obvious reasons, it is uncomfortable for a patient to keep eyes open during the proceedings.

Another major drawback of the DOL especially with reference to India is the ability to function in absence of basic essential commodity such as electricity. The problem is more prevalent in rural areas where most of the population resides. In absence of electricity, all these DOL technologies and other inventions for lighting oral cavity become irrelevant. An already available option is to set up power supply using a generator. This is a feasible at places where there are infrastructure to avail it, but where transient camps are installed; temporary arrangements are to be made for power supply.

In view of such circumstances the backup systems are economically not viable; moreover there is always a fear of over loading the system and starting a fire. Accordingly, there is a need of such devices which overcome the aforementioned shortcomings in the state of the art and provide a viable, economic and energy efficient solution for the same.

Although the use of chemi-luminance for illuminating the bucccal cavity is not new, however there are many limitation to the prior art.

U.S. Pat. No. 7,153,131, provides an intraoral illumination device for illuminating the interior of the mouth of a dental patient. The device comprises a flexible container having container defining walls, a portion of the walls being light transmitting. The device is sized for insertion into the mouth and placement against an interior surface of the mouth, e.g., the gum. A pressure-sensitive adhesive layer covers a portion of the walls for adhesively mounting the device to the interior surface of the mouth. A first chamber and a second chamber are within the container. The chambers are connected to each other by a frangible wall. Each of the chambers contain a chemical composition that when mixed together create a chemi-luminescent reaction that produces light. When the frangible wall is fractured by flexing the container, it permits the compositions to mix within the container to create a chemi-luminescent reaction that emits light through the container walls to illuminate the mouth, permitting dental procedures to be easily performed.

U.S. Pat. No. 6,299,441 B1, provides product includes a holder to be held inside the mouth of a user and a carrier to be held removably by the holder and having a first compartment containing a chemi-luminescent solution separated from a second compartment containing an activator for the chemi-luminescent solution by a frangible barrier. The carrier may be inserted into the holder and the holder flexed to break the barrier and mix the chemi-luminescent solution and activator to produce chemi-luminescent light inside the mouth of a user. The carrier can be replaced upon the light produced diminishing.

The problem with the prior art is that all of the inventions provide just one light source either in the centre or attached to the opposite end of the target, i.e. a device is placed with the help of an adhesive or a tubular strip inside the buccal cavity chemi-luminance illuminates the buccal cavity. However, as any surgeon shall specify that uniform and adequate DOL is the primary requirement i.e. there should be enough light that uniformly illuminates the buccal cavity from more than one source or direction, to eliminate the shadow formation while working.

Placing a tube in certain designated area does not compensate for uniform lighting, further if such placement is done in the middle or directly opposite to the place to be examined there shall be shadowing.

Further as dental procedures require sufficient time, therefore a support should be provided to open the buccal cavity for extended period of time.

There are other problems associated such as storage, as these are of very unusual shape and a non-flexible material; therefore storage to avoid breakage is a vital issue.

The device should facilitate multiple actions and functions such as a valve at the split junction should be provided, making it capable to attach a suction tube to facilitate saliva ejection and at least one LED diode—charged externally by a capacitor, operated via a wireless transmitter to sync the flash of the camera during intra-oral photography.

The device should be easy to clean, should allow saliva and other fluids to drain properly, adequately and quickly, without taking out the support or the retractor. There should be provisions to provides a detachable window for intra-oral camera/laser diode/photo-spectrometer for detecting lesions like leukoplakia and oral lichen planus which are known to have different refractive properties when a chemi-luminescent light is shined on such diseased tissue; and optionally a tongue guard with adhesive base chemi-luminescent pads should be provided to be attached on the undersurface of the tongue area for the dual purpose of isolation and illumination.

Other major problem that needs to be referred is the force exerted by the cheek retractor that exerts uncontrollable amount of force, to split the upper and lower jaw and maintain it in such position.

Therefore, there is a technological gap wherein a chemi-luminescent cheek retractor is required to overcome all the existing problems.

### OBJECT OF THE INVENTION

Accordingly, the main object of the invention is to provide a chemi-luminescent cheek retractor, with the entire cheek retractor (angular oris) including the handle(s) providing light from various angles and is directed by specialized coating to eliminate shadowing while performing a procedure.

Another object of the invention is to provide a method of creating chemi-luminescence in cheek retractor, wherein the method comprises allowing chemical reagents to make contact with triggering agents resulting in chemi-luminescence.

Yet another object of the invention is to provide a chemi-luminescent cheek retractor which is tamperproof, light weight, safe, reusable and easy to clean.

Yet another object of the invention is to provide the chemi-luminescent cheek retractor to produce adequate lighting with uniform intensity and high color rendering index (CRI).

Yet another object of the invention is to provide a chemi-luminescent cheek retractor to produce 2-4 hours of sustainable light.

Yet another object of the invention is to provide a chemi-luminescent cheek retractor with replaceable/refillable chemical reagents and triggering agents and is easily sterilized.

Yet another object of the invention is to provide a chemi-luminescent cheek retractor that has an angular oris lined with a reflective coating for providing uniform illumination.

Yet another object of the invention is to provide a chemi-luminescent cheek retractor that has one way pressure valves attached on the inlet of cheek retractor for triggering of chemi-luminescent reaction and inflating of cheek retractor.

Yet another object of the invention is to provide a chemi-luminescent cheek retractor attached with atleast one detachable extendable flange capable of attaching a suction tube to facilitate saliva ejection.

Yet another object of the invention is to provide a cheek retractor with an optional tongue guard with adhesive base chemi-luminescent pads provided to be attached on the underside of the tongue for the dual purpose of isolation and illumination.

Yet another object of the invention is to provide a cheek retractor having an angular oris with a detachable filter to provide the illumination at different wavelengths.

### SUMMARY OF THE INVENTION

The present invention provides a self-illuminating cheek retractor. More particularly, the invention relates to an improved apparatus for dental operatory light. The invention provides chemical luminescence within a cheek retractor for adequate lighting inside the oral cavity with uniform intensity and high color rendering index (CRI), without any external power source.

The present invention overcomes all drawbacks in state of the art technology, by providing a chemi-luminescent cheek retractor comprising a housing made of a transparent heat resistant material, an angular oris, a handle. The housing encloses a chemi-luminescent chamber extending from the angular oris to the handle. Further, the angular oris comprises an anterior transparent wall and a posterior wall lined with a reflective coating providing uniform illumination towards the oral cavity; the transparent heat resistant flexible material withstands the heat generated during the chemi-luminescent reaction; said chemi-luminescent cheek retractor is inflated with atleast one chemi-luminescent chemical reagent and triggered by atleast one triggering agent to trigger a chemic-luminescent reaction; said cheek retractor is inflated through a pressure valve for impelling the chemical reagents to reach an angular oris and then injecting the triggering agent to allow the chemi-luminescent reaction; a one way pressure valve is attached to an inlet of chemi-luminescence chamber of cheek retractor for triggering the chemi-luminescence reaction and for inflating cheek retractor; slots are provided on the chemi-luminescent cheek retractor for attaching detachable components. For instance a detcahable filter to provide illumination at selected wavelengths is attached to the angular oris attached to; and optionally a tongue guard with adhesive base chemi-luminscent pads to be attached on underside of tongue.

In an embodiment of the present invention, the cheek retractor has a continuous transparent heat resistant chamber filled with a chemi-luminescent reagent and injecting a triggering agent through pressure valve allows chemi-luminescence reaction. An angular oris is lined with a reflective coating to aid in better and uniform illumination, and directing the light towards the buccal cavity. The continuous chamber extends from angular oris to the handle(s). The illumination is not secluded to one part, rather the illumination is generated in chemi-luminescence chamber in angular oris and handle which is away from the patients buccal cavity, the internal reflective coating ensures that the illumination is more focused towards the cavity. An extended lighting source also ensures that shadowing is minimized due to multiple sources of light and overlapping fields of illumination.

The invention thus provides a cheek retractor that does not require external power source and is economically viable. The cheek retractor lighting system provides 2-4 hours of luminance. The perishable chemical reagents, in any form, can be easily replaced so the device can be used incessantly after disinfection.

In yet another embodiment the cheek retractor is optionally a tongue guard with adhesive base chemi-luminescent pads, provided to be attached on the underside of the tongue, for the dual purpose of isolation and illumination. The angular oris of the cheek retractor is detachably attached with a filter to provide illumination at select wavelengths.

### BRIEF DESCRIPTION OF THE DRAWINGS

A complete understanding of the system and method of the present invention may be obtained by reference to the following drawings:

FIG. 1 elucidates a preferred embodiment of the chemi-luminescent cheek retractor from the top view;

FIG. 2 elucidates a preferred embodiment of the chemi-luminescent cheek retractor from the top perspective view; and

FIG. 3 elucidates a preferred embodiment of the chemi-luminescent cheek retractor from the front perspective view.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

The present invention provides a self-illuminating cheek retractor. More particularly, the invention relates to an improved apparatus for dental operatory light. The invention provides chemical luminescence within a cheek retractor for adequate lighting inside the oral cavity with uniform intensity and high color rendering index (CRI), without any external power source.

The present invention overcomes all drawbacks in state of the art technology, by providing a chemi-luminescent cheek retractor comprising a housing made of a transparent heat resistant material, an angular oris, a handle. The housing encloses a chemi-luminescent chamber extending from the angular oris to the handle. Further, the angular oris comprises an anterior transparent wall and a posterior wall lined with a reflective coating providing uniform illumination towards the oral cavity; the transparent heat resistant flexible material withstands the heat generated during the chemi-luminescent reaction; said chemi-luminescent cheek retractor is inflated with atleast one chemi-luminescent chemical reagent and triggered by atleast one triggering agent to trigger a chemic-luminescent reaction; said cheek retractor is inflated through a pressure valve for impelling the chemical reagents to reach an angular oris and then injecting the triggering agent to allow the chemi-luminescent reaction; a one way pressure valve is attached to an inlet of chemi-luminescence chamber of cheek retractor for triggering the chemi-luminescence reaction and for inflating cheek retractor; slots are provided on the chemi-luminescent cheek retractor for attaching detachable components. For instance a detachable optical filter to provide illumination at selected wavelengths is attached to the angular oris attached to; and optionally a tongue guard with adhesive base chemi-luminscent pads to be attached on underside of tongue. The detachable filter is preferably an absorptive filter to transmit desired wavelengths by absorbing unwanted ones. These state of the art filters consist of dyed glass or pigmented gelatin resins. The ability of absorptive filters to attenuate light is based on the filter's physical thickness and the amount of dye or pigmentation present. The heat resistant flexible material comprises medical grade silicone in accordance with the present invention. As known in the art, one of the most important characteristics of silicone is its heat resistance, allowing silicone products to maintain their properties when exposed to both high and low temperatures.

In an embodiment of the present invention, the cheek retractor has a continuous transparent heat resistant chamber filled with a chemi-luminescent reagent and injecting a triggering agent through pressure valve allows chemi-luminescence reaction. An angular oris is lined with a reflective coating to aid in better and uniform illumination, and directing the light towards the buccal cavity. The continuous chamber extends from angular oris to the handle(s). The illumination is not secluded to one part, rather the illumination is generated in chemi-luminescence chamber in angular oris and handle which is away from the patients buccal cavity, the internal reflective coating ensures that the illumination is more focused towards the cavity. An extended lighting source also ensures that shadowing is minimized due to multiple sources of light and overlapping fields of illumination.

The invention thus provides a cheek retractor that does not require external power source and is economically viable. The cheek retractor lighting system provides 2-4 hours of luminance. The perishable chemical reagents, in any form, can be easily replaced so the device can be used incessantly after disinfection.

In yet another embodiment the cheek retractor is optionally a tongue guard with adhesive base chemi-luminescent pads, provided to be attached on the underside of the tongue, for the dual purpose of isolation and illumination. The angular oris of the cheek retractor is detachably attached with an optical filter to provide illumination at select wavelengths.

The chemi-luminescent cheek retractor for illuminating a buccal cavity comprises a housing, an angular oris (106) and a handle (108); the housing encloses a chemi-luminescence chamber (101) extending from the angular oris (106) to the handle (108), said housing is made of a flexible heat resistant material; the angular oris (106) comprises an anterior transparent wall (106a) and a posterior wall (106b) lined with a reflective coating; said angular oris (106) is attached to a detachable filter (111) to provide illumination at selected wavelengths; the handle comprises a one way pressure valve at an inlet (109), and slots (104) for attaching detachable components of the cheek retractor; said cheek retractor is inflatable with atleast one chemi-luminescent chemical reagent when injected through the one way pressure valve for impelling the chemi-luminescent chemical reagent to reach the chemi-luminescence chamber (101); the cheek retractor is illuminated by a chemi-luminescence reaction, said chemi-luminescence reaction is triggered with atleast one triggering agent injected through the one way pressure valve; and the cheek retractor optionally has a tongue guard (107) with adhesive base having pads attached on underside of tongue.

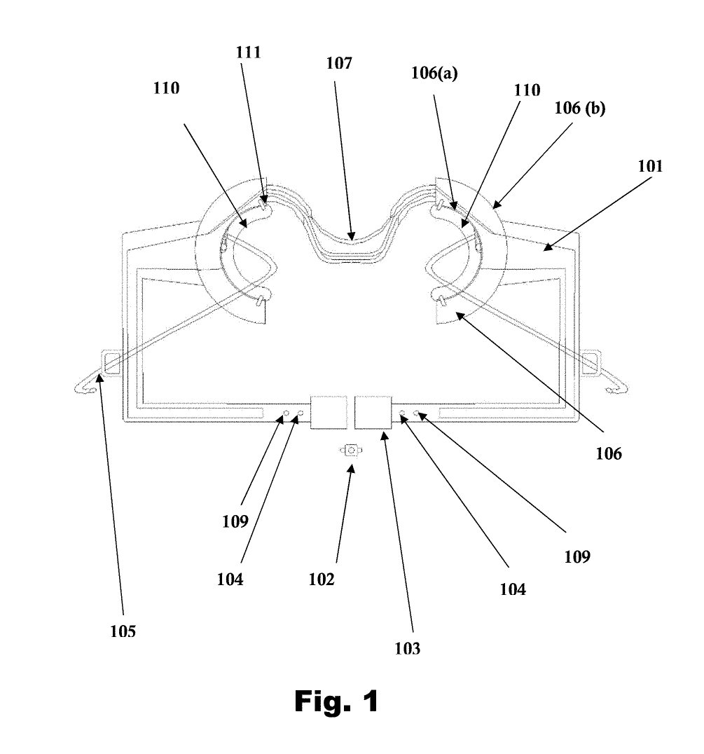

FIG. 1, elucidates the preferred embodiment of a novel chemi-luminescent cheek retractor wherein Chemi-luminescence chamber 101 is not localized to one side, instead it runs through a handle 108 and an angular oris 106. Further, Chemi-luminescence chamber 101 is made of a transparent material to withstand the heat generated during the chemi-luminescence reaction that extends from angular oris 106 to the handle 108. The angular oris 106 having anterior wall 106a and posterior wall 106b. The posterior wall 106b is lined with a reflecting coating to focus the illumination towards buccal cavity and minimizing shadowing, by overlapping the field of illumination. The attachment knob 102 allows gadgets or any attachments inserted into the fastening groove 103 to be secured properly, making it more versatile. Multiple attachments are possible or even adjustments can be made due to, extra attachment slots 104. The inlet 109 on the cheek retractor is for fixing the pressure valve thus inflating the cheek retractor with chemical reagents and injecting with triggering agent to allow a chemi-luminescence reaction. The chemical reagents are in either solid, liquid, or gas form or any combination such as divinyle oxalate, 1.0 M NaoH, hydrogen peroxide, a fluorescer (eg. Perylene) and triacetin as solvent. The triggering agents are in either solid, liquid, or gas form such as ozone, hydrogen peroxide and alike. The cheek retractor is inflatable through pressure valve to impel the chemical reagents to reach the angular oris 106. The angular oris is attached to a detachable filter to provide illumination at selected wavelengths. The cheek retractor is further attached to a detachable filter 110 through a snap lock 111 to provide illumination at different/selected wavelengths.

Further, at least one detachable extendable flanges are provided 105 to abet attachments for aid in isolation by providing a channel for removing saliva. At least one tongue away shield 107 is provided that folds or keeps the tongue away during procedures and therefore, minimizes accidents.

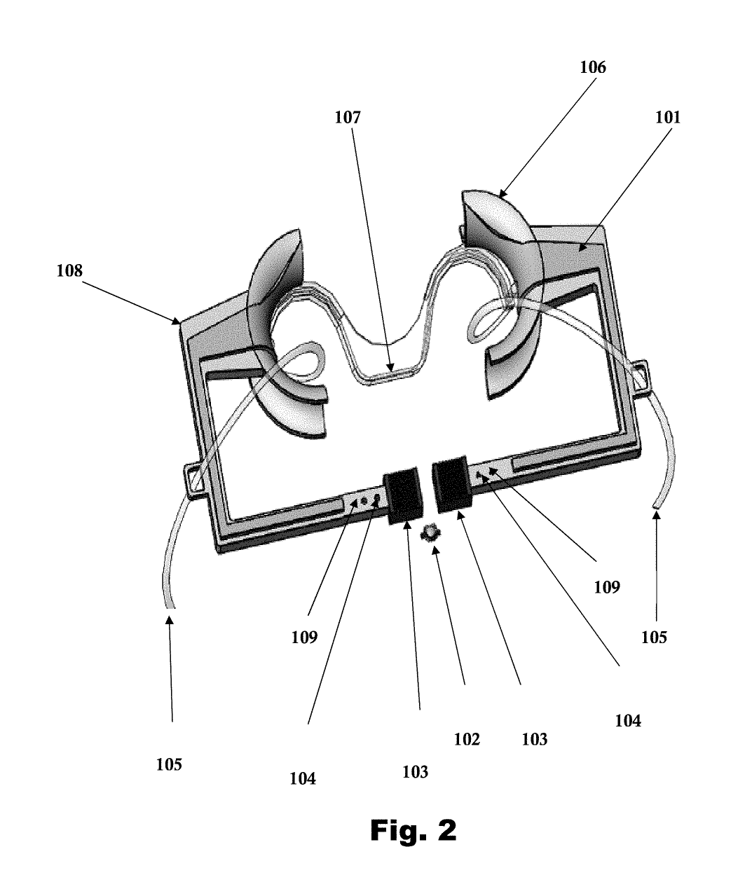

Further illustration of the preferred embodiment is shown in FIG. 2 elucidates the preferred embodiment of the chemi-luminescent cheek retractor from the top perspective view.

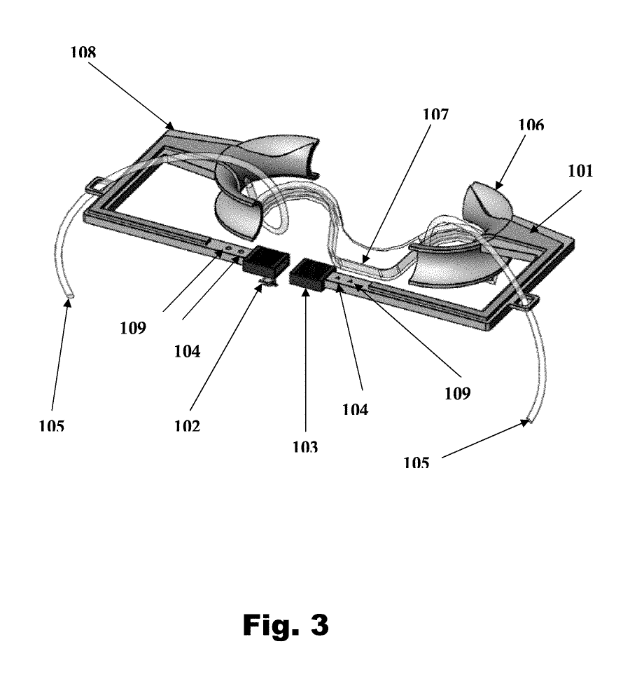

FIG. 3 elucidates the preferred embodiment of the chemi-luminescent cheek retractor from the front perspective view.

While the best mode has been described in detail, those familiar with the art will recognize various alternative designs and examples within the scope of the following claims.

We claim:

1. A chemi-luminescent cheek retractor for illuminating a buccal cavity comprising a housing, an angular oris (106) and a handle (108); wherein:
the housing encloses a chemi-luminescence chamber (101) extending from the angular oris (106) to the handle (108), said housing is made of a flexible heat resistant material;
the angular oris (106) comprises an anterior transparent wall (106a) and a posterior wall (106b) lined with a reflective coating;
said angular oris (106) is attached to a detachable filter (111) to provide illumination at selected wavelengths;
the handle comprises a one way pressure valve at an inlet (109), and slots (104) for attaching detachable components of the cheek retractor;
said cheek retractor is inflatable with atleast one chemi-luminescent chemical reagent when injected through the one way pressure valve for impelling the chemi-luminescent chemical reagent to reach the chemi-luminescence chamber (101);
the cheek retractor is illuminated by a chemi-luminescence reaction, said chemi-luminescence reaction is triggered with atleast one triggering agent injected through the one way pressure valve; and
the cheek retractor optionally has a tongue guard (107) with adhesive base having pads attached on underside of tongue.

2. The chemi-luminescent cheek retractor as claimed in claim 1, wherein the transparent heat resistant material includes silicone based material and withstands the heat generated during chemi-luminescent reaction.

3. The chemi-luminescent cheek retractor as claimed in claim 1, wherein the atleast one chemi-luminescent chemical reagent is in solid, liquid, gas form or any combination such as divinyle oxalate, 1.0 M NaoH, hydrogen peroxide, a fluorescer (eg. Perylene) and triacetin as solvent.

4. The chemi-luminescent cheek retractor as claimed in claim 1, wherein the atleast one triggering agent is in solid, liquid, gas form such as ozone, hydrogen peroxide and alike.

5. The chemi-luminescent cheek retractor as claimed in claim 1, wherein, at least one detachable extendable flange (105) is provided to abet attachments for aid in isolation by providing a channel for removing saliva.

6. The chemi-luminescent cheek retractor as claimed in claim 1, wherein, an attachment knob (102) is provided for fastening detachable components in grooves (103) on the chemi-luminescent cheek retractor.

7. The chemi-luminescent cheek retractor as claimed in claim 1, wherein, said reflective coating focuses illumination towards buccal cavity.

8. The chemi-luminescent cheek retractor as claimed in claim 1, wherein, said chemi-luminescence reaction illuminates the buccal cavity for 2-4 hours.
